(19) United States
(12) Patent Application Publication
(10) Pub. No.: US 2001/0053931 A1
(43) Pub. Date: Dec. 20, 2001

(54) THIN-LAYERED, ENDOVASCULAR SILK-COVERED STENT DEVICE AND METHOD OF MANUFACTURE THEREOF

(75) Inventors: KATHY HESS, MIDDLETON, MA (US); BARBARA KELLEY, BEDFORD, MA (US)

Correspondence Address: HOFFMANN & BARON LLP, 6900 JERICHO TURNPIKE, SYOSSET, NY, 11791

(73) Assignee: Salvatore J. Abbruzzese

(21) Appl. No.: 09/448,701

(22) Filed: Nov. 24, 1999

(51) Int. Cl. A61F 2/06

(52) U.S. Cl. 623/1.15

(57) ABSTRACT

A stent-graft composite intraluminal prosthesis comprises an elongate radially adjustable tubular stent, defining opposed exterior and luminal stent surfaces and a polymeric stent sheath covering at least the exterior surface thereof. The stent can include a plurality of open spaces extending between the opposed exterior and interior surfaces so as to permit said radial adjustability. The stent has a polymeric material on its exterior surface, its interior surface, in interstitial relationship with the stent or any combination of the above. The polymer is preferably selected from the group of polymeric materials consisting of biological or genetically engineered spider silks, such as those derived from Nephila clavipes. The silk includes bioengineered spider silks as well as silk-like polymers manufactured using human proteins and blends of such silks with commonly used polymeric graft materials. If separate sheaths are placed on both the exterior and interior surfaces of the stent, the sheaths are secured to one another through said open spaces, such as by lamination, suturing or adhesion.

### FIELD OF THE INVENTION

The present invention relates generally to a tubular implantable prosthesis including a stent and graft composite structure used to repair and/or replace or otherwise treat a body vessel. More particularly, the present invention relates to a stent-graft composite device including a radially expandable stent employing natural or bioengineered spider silk or its derivatives as a covering.

### BACKGROUND OF THE INVENTION

Employment of various implantable tubular prostheses in medical applications is well known for the treatment of a wide array of vascular and other diseases. Such tubular prostheses are used extensively to repair, replace or otherwise hold open blocked or occluded body lumens such as those found in the human vasculature.

One type of prosthesis which is especially useful in maintaining the patency of a blocked or occluded vessel is commonly referred to as a stent. A stent is a generally longitudinal tubular device formed of biocompatible material which is useful in the treatment of stenosis, strictures or aneurysms in body vessels such as blood vessels. These devices are implanted within a vessel to reinforce collapsing, partially occluded, weakened or abnormally dilated sections thereof. Stents are typically employed after angioplasty of a blood vessel to prevent re-stenosis of the diseased vessel. While stents are most notably used in blood vessels, stents may also be implanted in other body vessels such as the urogenital tract and bile duct.

Stents are generally radially expandable tubular structures which are implanted intraluminally within the vessel and deployed at the occluded location. A common feature of stent construction is the inclusion of an elongate tubular configuration having open spaces therethrough which permit radial expansion of the stent. This configuration allows the stent to be flexibly inserted through curved vessels and further allows the stent to be radially compressed for intraluminal catheter implantation. Flexibility is a particularly desirable feature in stent construction as it allows the stent to conform to the bends in a vessel.

Once properly positioned adjacent the damaged vessel, the stent is radially expanded so as to support and reinforce the vessel. Radial expansion of the stent may be accomplished by inflation of a balloon attached to the catheter, or the stent may be of the self-expanding variety which will radially expand once deployed. Structures which have been used as intraluminal vascular grafts have included coiled stainless steel springs; helically wound coil springs manufactured from a heat-sensitive material; and expanding stainless steel stents formed of stainless steel wire in a zig-zag pattern. Examples of various stent configurations are shown in U.S. Pat. No. 4,503,569 to Dotter; U.S. Pat. No. 4,733,665 to Palmaz; U.S. Pat. No. 4,856,561 to Hillstead; U.S. Pat. No. 4,580,568 to Gianturco; U.S. Pat. No. 4,732,152 to Wallsten and U.S. Pat. No. 4,886,062 to Wiktor.

Another implantable prosthesis which is commonly used in the vascular system is a vascular graft. Grafts are elongate tubular members typically used to repair, replace or support damaged portions of a diseased vessel. Grafts exhibit sufficient blood tightness to permit the graft to serve as a substitute conduit for the damaged vessel area.

The most important features of a graft are porosity, compliance and biodegradability. A graft should be microporous to provide a stable anchorage for vascular cells and stimulate tissue ingrowth and cell endothelialization therealong. Porosity is an essential component for functional synthetic vascular prostheses and plays an important part in their long-term patency. Grafts which are impermeable to blood after the time of implantation do not permit the subsequent ingrowth of cells which is necessary for uniform and satisfactory bonding of the internal lining of a prosthesis.

In addition, the graft should be compliant to stimulate ingrowing tissue and form a new elastic component of a vascular or other lumen. Poor compliance is one of the most important factors responsible for the poor performance of synthetic vascular grafts. Poor compliance prevents the reconstruction of narrow lumens by causing occlusions in the replacement prosthesis. A mismatch in compliance between the lumen and the graft results not only in high shear stress, but also in turbulent blood flow with local stagnation.

The graft may also be biodegradable so that the ingrowing tissue can take over the function of the graft. This improves the patency of the graft and promotes long term healing.

Vascular grafts may be fabricated from a multitude of materials, such as synthetic textile materials and fluoropolymers (i.e. expanded polytetrafluoroethylene (ePTFE)) and polyolefinic material such as polyethylene and polypropylene. Nylon is often used, but polyester is chosen more frequently because of its good mechanical and chemical properties. Polyester is the most commonly used because it is available in a wide range of linear densities and its low moisture absorption also gives good resistance to fast deterioration. Polyurethane is another polymer especially used for its elasticity. Graft material selection is not limited to those materials listed above, but may include others that are conducive to the biocompatibility, distensibility and microporosity requirements of endovascular applications.

If the graft is thin enough and has adequate flexibility, it may be collapsed and inserted into a body vessel at a location within the body having diameter smaller than that of the intended repair site. An intraluminal delivery device, such as a balloon catheter, is then used to position the graft within the body and expand the diameter of the graft therein to conform with the diameter of the vessel. In this manner, the graft provides a new blood contacting surface within the vessel lumen. An example of a graft device as described herein is provided in commonly assigned U.S. Pat. No. 5,800,512 to Lentz et al.

Composite stent-graft devices employing tubular structures are also known wherein a stent is provided with one or both of a polymeric cover disposed at least partially about the exterior surface of the stent and a polymeric liner disposed about the interior surface of the stent. These composite devices have the beneficial aspects of a stent, which is used to hold open a blocked or occluded vessel, and also a graft which is used to replace or repair a damaged vessel. Several types of stent-graft utilize fibrous grafts having porosity conducive to tissue ingrowth and elasticity conducive to expansion and contraction within a fluid environment. Often, fibers of various materials are used, alone or in combination, to form graft structures that accentuate the positive effects of stents on their vascular environment. Use of fibers obviates the need to shape and mold a device into its ultimate working configuration, and many fibers have proven to be biocompatible with vascular tissues.

Several types of stent-graft devices are known in the art. Examples of such stent-graft composite devices are shown in U.S. Pat. No. 5,476,506 to Lunn; U.S. Pat. No. 5,591,199 to Porter et al.; U.S. Pat. No. 5,591,223 to Lock et al.; and U.S. Pat. No. 5,607,463 to Schwartz et al.

The procedures which utilize the above disclosed devices obviate the need for major surgical intervention and reduce the risks associated with such procedures. While such composite devices are particularly beneficial due to the thinness at which they may be formed and the radial strength which they exhibit, the devices may suffer from a lack of biocompatibility in long-term applications, such as those in which therapeutic drugs are to be delivered over an extended period of time. Thus, it may be difficult to maintain an endovascular device having graft materials formed from polymeric materials that induce inflammatory responses in native vessels.

Reduction of implantation-related inflammation can be effected by selection of graft materials that are inherently more biocompatible than those heretofore employed in stent-graft devices. Conventional graft materials such as PET polyester and nylon have high solubility factors which indicate that the material is prone to higher rates of solubilization within native vessels and therefore more prone to inflammatory responses. Such responses can translate in swelling of the surrounding vessel and impeded blood flow therethrough as a result thereof. Inflammations can further lead to tissue ingrowth at the periphery of the prosthesis, further impeding blood flow and defeating the purpose of the stent-graft device to not only maintain the patency of the vessel, but also assist in the healing of surrounding tissue.

Biological or bioengineered silk material, on the other hand, exhibits desirable characteristics which inhibit the inflammatory responses observed with other conventional polymeric materials used in stent-graft applications. Woven silk material possesses a smooth surface which does not interfere with the inherent hemodynamic properties of blood flow. Biological silks also have natural elastic properties that increase endoprosthetic distensibility over conventional stent-graft materials.

Biological silks are typically derived from silkworms. Fibers produced by silkworms can be easily fabricated into cloth, however, the strength and toughness of silkworm silk is relatively low. Because silkworm fibers are too fine for commercial use, between 3 and 10 strands are used at a time to achieve a silk strand of required diameter for weaving.

Spider silk, however, demonstrates superior mechanical properties which make it desirable in use for various medical applications, including stent-graft endoprostheses. The combined high tensile strength (4×10⁹ N/m²) and elasticity (35%) of major ampullate spider silk (also known as “dragline” silk) translates into a toughness that is superior to all man-made or natural fibers, including silkworm strands. The silk is thus five times stronger than steel, yet 30% more flexible than nylon and can absorb three times the impact force without breaking than Kevlar.

An orb web, the typical spider web, is constructed of several different silk types, each composed primarily of protein. These silks vary in their mechanical properties over a very wide range of tensile strength and elasticity. The best studied silk is dragline silk from Nephila clavipes, also known as the golden orb weaving spider. This one spider can synthesize as many as six types of silk, each having slightly different mechanical properties. Dragline silk is a semicrystalline polymer which, besides forming the dragline, is used to form the frame of the web. The material must perform functions such as absorbing the energy of a flying insect so that the prey neither breaks nor bounces off of the trap. Dragline silk must also support the weight of a rapelling spider. Dragline silk is stronger than a steel cable of the same diameter.

In addition, dragline silk is the only silk that has the ability to supercontract. Wetting of unrestrained fibers of dragline silk at room temperature causes the fibers to contract to about 60% of their relaxed dry length. In synthetic fibers, such supercontraction occurs only at extreme temperatures or in harsh solvents. Supercontraction of dragline fibers is accompanied by a decrease in tensile strength and an increase in elongation before breaking. Unlike synthetic fibers, however, the mechanical properties of the dragline fiber return to their original values once dried and re-stretched.

Furthermore, dragline silk is also non-allergenic, making it very desirable for medical applications. Single strands are only 1/20,000 of an inch across apiece, and the diameter of the fiber ranges from 0.1 to 8μ, depending upon the type of silk. Spider silk is a soluble fluid in the aqueous environment of the spider's abdomen, but it is an insoluble solid after it exits the spider's body. Insolubility is a major factor in a web's durability which can translate into an increased lifespan for endoprosthetic devices.

Synthetic genes can be designed to encode analogs of the silk proteins to produce biosilk. Use of recombinant DNA technology enables bacteria to produce and customize the silk proteins which form silk substances. Silk-like protein polymers can also be implemented, such as ProNectin F (a trademark of Protein Polymer Technologies of San Diego, Calif.). Such polymers mimic the molecular structure of natural silk and incorporate properties of human proteins. They can be processed in films and bond with different types of cells in native tissue.

The following table presents a comparison of extensibility and tensile strength of three spider silks of Nephila clavipes:

| Silk | Extension | Tensile Strength |
|---|---|---|
| Major ampullate (dragline) | 35% | 400 kpsi |
| Minor ampullate | 5 | 100 |
| Flagelliform | 200 | 100 |

Thus, endoprosthetic devices which employ spider silk and derivatives thereof (i.e. biosilk, combinations of silk/biosilk with other well-known polymeric graft materials) would not only retain their shape better, but also remain more flexible. In addition, because the proteins which form the silk substances comprise a biological material, they integrate more effectively in the human body.

Accordingly, it is desirable to implement a biosilk material, either naturally occurring or genetically engineered, in a stent-graft device which exhibits sufficient radial strength to permit the composite device to accommodate a radially expandable stent and yet improves biocompatibility with a vascular site into which implantation occurs. It is further desirable to provide an expandable tubular stent which exhibits sufficient radial strength to permit the stent to maintain patency in an occluded vessel and yet prevent reoccurrence of occlusions in a passageway by providing an expandable tubular stent of generally open, cylindrical configuration that utilizes silk material. Such a device prevents inflammation of lumen passageways due to incompatibility with graft material and assists in the healing of diseased lumen tissue by enabling extended elution of therapeutic substances therefrom.

### SUMMARY OF THE INVENTION

It is an advantage of the present invention to provide an improved tubular stent-graft composite device.

It is another advantage of the present invention to provide an easily manufactured stent-graft device which reduces tissue inflammation due to implantation of the device within vascular tissue.

It is yet another advantage of the present invention to provide a stent-graft composite device having the dual function of structural support for a radially expandable stent and absorption and release of therapeutic agents.

It is a further advantage of the present invention to utilize biological silk substances as graft coverings to assist blood flow and reduce inflammatory reactions in stent-graft endoprostheses.

The present invention provides a stent-graft composite intraluminal prosthesis comprising an elongate radially adjustable tubular stent, defining opposed interior and exterior stent surfaces and a polymeric stent sheath covering at least the exterior surface of the stent. The stent can include a plurality of open spaces extending between the opposed exterior and interior surfaces so as to permit said radial adjustability. The stent has a polymeric material on its exterior surface, its interior surface, in interstitial relationship with the stent or any combination of the above. The polymer is preferably selected from the group of polymeric materials consisting of biological or genetically engineered spider silks, such as those derived from Nephila clavipes. If separate sheaths are placed on both the exterior and interior surfaces of the stent, the sheaths are secured to one another through said open spaces, such as by lamination, suturing or adhesion. One of the sheaths may comprise a tubular structure fabricated from a conventional polymeric graft material, such as polyester or nylon. In the alternative, either tubular sheath may include a combination of biological or bioengineered spider silk with polymeric fibers.

A method of making a stent-graft endovascular prosthesis of the present invention is also disclosed. The disclosed method includes providing an elongated radially adjustable tubular stent, defining opposed interior and exterior stent surfaces. A tubular silk structure is disposed about at least one of an exterior and luminal surface of the stent and secured thereto. Securement is effected preferably by sutures, however, when both the exterior and luminal stent surfaces are to be covered, the silk structures may be secured through the open spaces of the stent as described hereinabove. If separate sheaths are placed on both the exterior and interior surfaces of the stent, the sheaths are secured to one another through said open spaces, such as by sutures also made from a spider silk or derivative thereof.

### BRIEF DESCRIPTION OF THE DRAWINGS:

FIG. 1 is a perspective view of a preferred embodiment of a tubular stent-graft prosthesis of the present invention.

FIG. 2 is a perspective view of one embodiment of a stent which may be used in a stent-graft composite prosthesis of the present invention.

FIG. 3 shows a side view of a tubular stent-graft prosthesis of FIG. 1 having sutures therein.

FIG. 4 shows a cross-section of a preferred embodiment of the tubular stent-graft prosthesis of the present invention taken along line a-a of FIG. 3.

FIG. 5 shows a schematic of a polymeric film on a mandrel prior to affixing a stent thereon.

FIG. 6 shows a schematic of the film and mandrel of FIG. 5 after placement of a stent thereover.

FIG. 7 shows a cross-section of the stent and polymer combination of FIG. 6 after removal from the mandrel, taken along line b-b.

FIG. 8 shows a cross-section of the stent and polymer combination of FIG. 7 having a tubular silk structure disposed about an exterior surface thereof.

### DETAILED DESCRIPTION OF THE PREFERRED EMBODIMENTS

In the present invention, a tubular stent-graft prosthesis is provided which incorporates a tubular radially adjustable stent having a polymeric covering over an exterior and/or luminal surface thereof. The preferred covering is formed from biological or genetically engineered silk fibers such as those derived from spiders, or from fibers incorporating said silk and a polymeric graft material therein. Silk is a preferred covering because it is very biocompatible, has a smooth surface finish and has natural elastic properties that increase its distensibility over conventional stent-graft materials. The silk is employed as graft material for a stent wherein the material can be applied luminally, externally or laminated to the stent. The covering can either be flush with the ends of the stent or centered mid-stent, allowing a portion of the stent to remain uncovered. The covering can be secured to the stent using sutures, preferably also formed of silk.

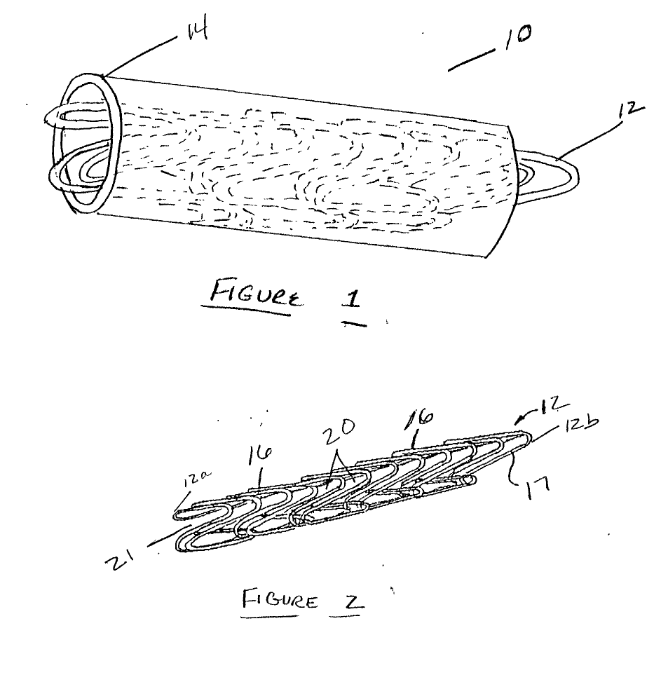

Now referring to the figures, where like elements are identically numbered, FIG. 1 shows a preferred embodiment of a tubular stent-graft prosthesis 10 of the present invention. . Prosthesis 10 includes a tubular radially expandable stent 12 having a sheath 14 on at least an exterior surface thereof. Sheath 14 includes a thin-walled material, preferably having a thickness between 0.005″-0.006″, inclusive. The sheath is made from a film or weave of silk or silk-like material such as spider dragline silks, bioengineered equivalents or combinations thereof (collectively referred to herein as “silk”) which are more biocompatible with vascular tissue than conventional graft materials. Silk material is selected because its remains insoluble in native vessels and therefore promotes a more biofriendly reaction when compared to current materials such as PET polyester and nylon. Currently utilized materials such as these exhibit a high solubility factor (10.7), resulting in an exacerbated inflammatory response in lumen tissue which in turn inhibits the effect of therapeutic substances placed thereon.

The silk material that is used in the device may have any of a variety of textures and finishes which promote endotheliazation. Such finishes includes smooth finishes that facilitate laminar blood flow and mesh-like material having improved porosity so as to promote endothelial lining/tissue growth. Blends of silk and polymers in the form of drawn fibers can also be used, as they exhibit an increased elastic modulus and moisture absorption factor which enables the prosthesis to thereby sustain tissue ingrowth thereon.

Although a wide variety of stents may be used, FIG. 2 shows a perspective view of one particular stent which may be employed in prosthesis 10. The particular stent shown in FIG. 2 is more fully described in commonly assigned U.S. Pat. No. 5,575,816 to Rudnick, et al. Stent 12 is an intraluminally implantable stent formed of helically wound wire. Multiple windings 16 of a single metallic wire 17, preferably composed of a temperature-sensitive material such as Nitinol, provide stent 12 with a generally elongate tubular configuration which is radially expandable after implantation in a body vessel. The multiple windings 16 of stent 12 define open spaces 20 throughout the tubular configuration and define a central open passage 21 therethrough between opposing extremities 12a and 12b. The helically wound wire configuration not only ensures patency and flexibility, but the open spaces also allow adhesion of tubular layers therethrough.

Although this particular stent construction is shown and described with reference to the present invention, various stent types and stent constructions may be employed in the present invention for the use anticipated herein. Among the various stents useful include, without limitation, self-expanding stent and balloon expandable stents. The stents may be capable of radially contracting as well, and in this sense can be best described as radially distensible or deformable. Self-expanding stents include those that have a spring-like action which causes the stent to radially expand or stents which expand due to the memory properties of the stent material for a particular configuration at a certain temperature. Nitinol is one material which has the ability to perform well while both in spring-like mode as in a memory mode based on temperature. Other materials are of course contemplated, such as stainless steel, platinum, gold, titanium and other biocompatible materials, as well as polymeric stents.

The configuration of the stent may also be chosen from a host of geometries. For example, wire stents can be fastened in a continuous helical pattern, with or without wave-like forms or zig-zags in the wire, to form a radially deformable stent. Individual rings or circular members can be linked together such as by struts, sutures, or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like.

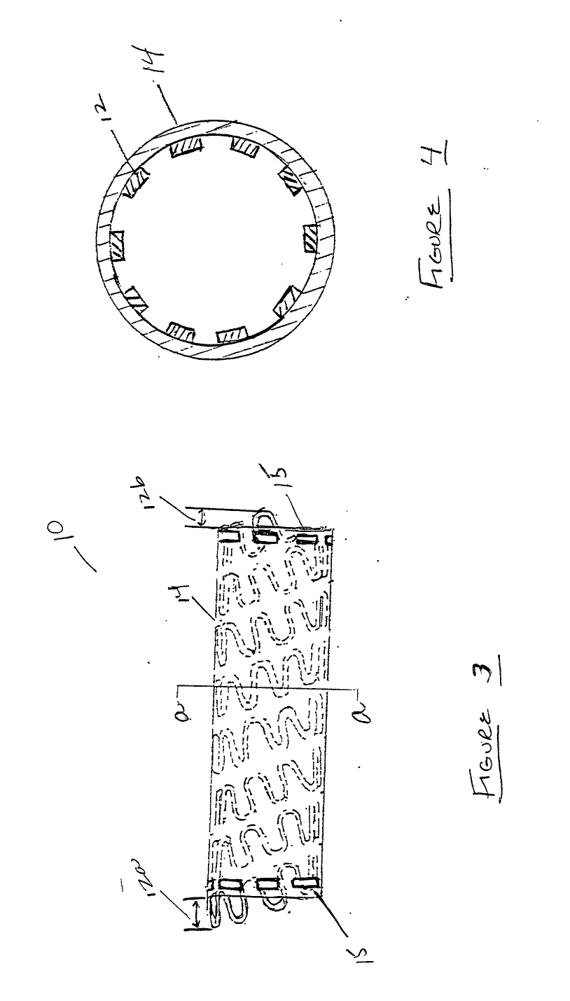

The fabrication of a composite device of the type shown in FIG. 1 can now be described. Prosthesis 10 is formed by providing a stent 12 with at least one silk tubular sheath 14 disposed circumferentially about an exterior surface thereof. As shown in FIG. 3, the silk sheath can either be flush with the ends of the stent or centered mid-stent allowing a small amount (i.e. approximately 2-3 mm) of open stent on both the proximal and distal stent extremities 12a and 12b . The exposed portions may be desirable in certain applications to ensure securement of the prosthesis after deployment to a repair site.

The covering itself can be applied to the stent in three different orientations which are external, internal, or laminated to the stent. Silk fibers or films can be attached to stent platforms by suturing the material to the stent as shown in FIG. 3. To suture the polymeric fiber or film to the stent, the preferred method is to use silk sutures 15 and attach sheath 14 to stent 12 at the sheath's distal and proximal ends. The number of sutures 15 that will hold the tubular silk material to the stent will depend on the stent diameter.

Sutures 15 can likewise be fabricated from spider silk, biosilk and derivatives or combinations thereof. Such sutures are one tenth the diameter of current silk sutures, reducing the amount of bleeding and scarring associated with surgical procedures. Although silk is the preferred suture material, other polymeric materials may be selected from the group consisting of absorbable (i.e., catgut, reconstituted collagen, polyglycolic acid) and nonabsorbable (i.e., silk, cotton and linen, polyester, polyamide, polypropylene and carbon fiber) materials. External factors that govern the selection of suture material include tissue type, temperature, pH, enzymes, lipids and bacteria.

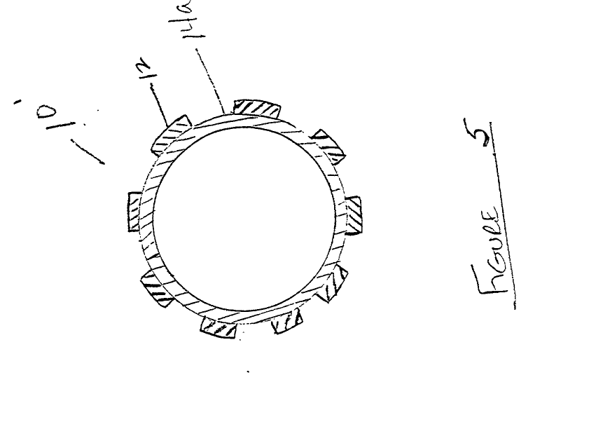

As is evident from FIG. 4, a cross section of prosthesis 10 reveals that sheath 14 circumferentially envelops the outer periphery of stent 12. Although sheath 14 appears as a substantially complete tube that is slid over the stent while on the mandrel 22, it is evident that the sheath may be a film or sheet having its opposing edges overlapped and secured to one another to form a tubular structure. It is anticipated that a luminal covering 14a can be similarly affixed to stent 12 as heretofore described and as illustrated in FIG. 5.

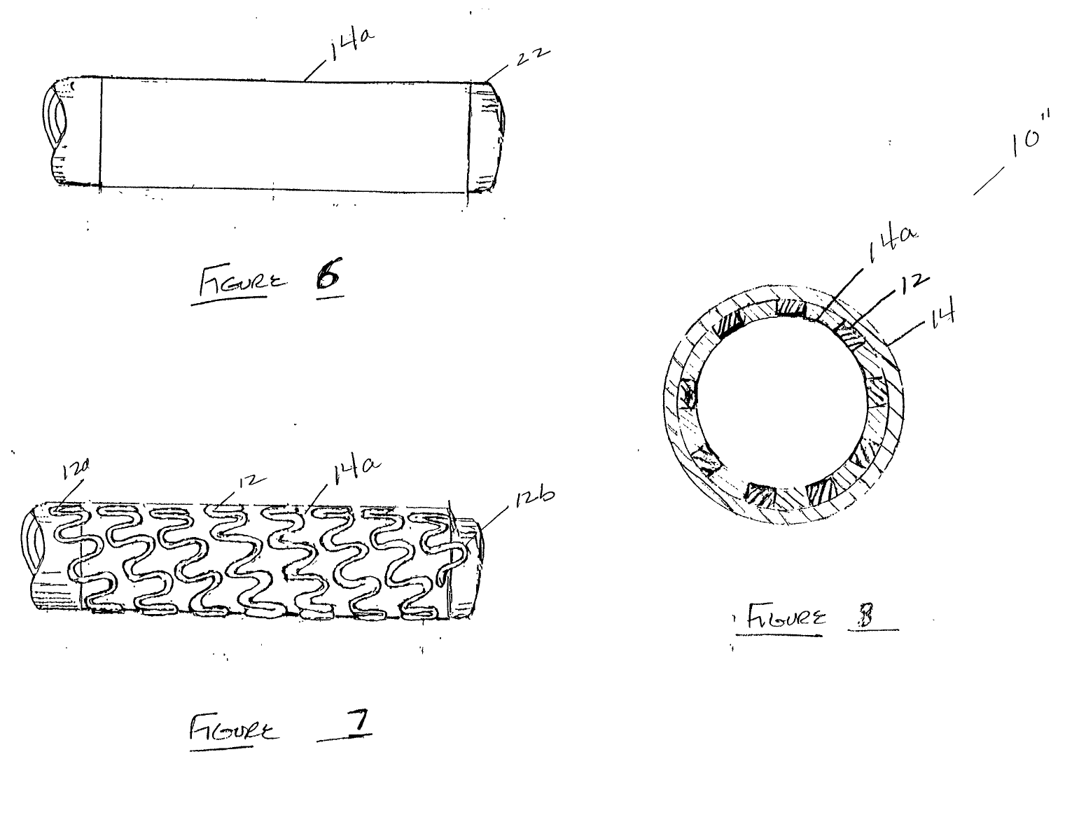

Sheaths 14 and 14a can be simultaneously applied to stent 12 to provide a prosthesis having dual graft coverings. One or both of sheaths 14 and 14a may be formed from a silk or silk derivative as described hereinabove. One of said sheaths may alternatively be formed form a polymeric material such as conventional PET polyester, nylon, polyethylene, polypropylene, polyurethane or combinations of any of these materials with one another or with the silk materials described herein. Referring to FIGS. 6 and 7, a polymeric sheath can serve as a sheath 14a that is provided on a mandrel 22 and has stent 12 affixed thereover. The mandrel and stent can then be placed into an oven for a time sufficient for sheath 14a to be inextricably melted within the open spaces of stent 12. Upon removal of the stent and sheath combination, a silk sheath 14 is placed thereover. A cross-section of this assembly is provided in FIG. 8. It is evident that a polymeric sheath can easily be provided on an exterior surface of stent 12 as well, with a silk sheath on a luminal surface of the stent.

Either or both of the luminal and exterior surface sheaths 14 and 14a may be provided with an adhesive thereon which permits adherence of the tubular structures to one another through the stent openings and simultaneously allows adherence of stent 12 to either or both of the structures. The adhesive may be a thermoplastic adhesive and more preferably, a thermoplastic fluoropolymer adhesive such as FEP. A suitable adhesive provides a substantially sealed tube without significantly reducing longitudinal and axial compliance.

The present prosthetic materials can also be implemented in an implantable vascular prosthesis or graft. “Vascular graft” can mean conventional and novel artificial grafts made of this material constructed in any shape including straight, tapered or bifurcated and which may or may not be reinforced with rings, spirals or other reinforcements and which may or may not have one or more expandable stents incorporated into the graft at one or both ends or along its length. The vascular graft of choice may be introduced into the vessel in any suitable way including, but not limited to, use of a dilator/sheath, placement of the graft upon a mandrel shaft and/or use of a long-nose forceps. The distal ends of the tubular graft and the mandrel shaft may be temporarily sutured together, or the distal end of the vascular graft may be sutured together over the mandrel to accommodate unitary displacement into a vessel, for example, through a sheath after the dilator has been removed. One or both ends of the vascular graft may be sutured or surgically stapled in position on the treated vessel to prevent undesired displacement or partial or complete collapse under vascular pressure.

Where the graft is expandable and in tubular or sleeve form, the diametrical size of the graft may be enlarged in contiguous relationship with the inside vascular surface via a balloon catheter. The tubular graft itself may comprise a biologically inert or biologically active anti-stenotic coating applied directly to the treated area of the remaining vascular inner surface to define a lumen of sufficient blood flow capacity. The graft, once correctly positioned and contiguous with the interior vascular wall, is usually inherently secure against inadvertent migration within the vessel due to friction and infiltration of weeping liquid accumulating on the inside artery wall. The length of the vascular graft preferably spans beyond the treated region of the vessel.

Additionally, the present invention prosthesis can be coated with hydrophilic or drug delivery type coatings which facilitate long-term healing of diseased vessels. The silk material can be loaded or coated with a therapeutic agent or drug, including, but not limited to, antiplatelets, antithrombins, anti-inflammatories, cytostatic and antiproliferative agents, for example, to reduce or prevent restenosis in the vessel being treated. The therapeutic agent or drug is preferably selected from the group of therapeutic agents or drugs consisting of sodium heparin, low molecular weight heparin, hirudin, prostacyclin and prostacyclin analogues, dextran, glycoprotein IIb/IIIa platelet membrane receptor antibody, recombinant hirudin, thrombin inhibitor, calcium channel blockers, colchicine, fibroblast growth factor antagonists, fish oil, omega 3-fatty acid, histamine antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDGF antagonists, alpha-interferon and genetically engineered epithelial cells, and combinations thereof. While the foregoing therapeutic agents have been used to prevent or treat restenosis and thrombosis, they are provided by way of example and are not meant to be limiting, as other therapeutic drugs may be developed which are equally applicable for use with the present invention.

The stent-graft prosthesis of the present invention features a variety of characteristics to make its widespread application efficacious, such as easy handling, suturability, capacity for uniform mass production, shelf storage, repeated sterilization and availability in appropriate sizes. Although various changes and modifications can be made to the present invention, it is intended that all such changes and modifications come within the scope of the invention as set forth in the following claims.

What is claimed is:

1. An improved implantable stent-graft prosthesis for minimizing tissue inflammatory responses, comprising:
an elongate radially adjustable stent having a substantially tubular configuration defining a central open passage therethrough, said stent having proximal and distal extremities and opposed interior and exterior stent surfaces wherein said stent includes plural open spaces extending between said opposed luminal and exterior surfaces so as to permit said radial adjustability;
at least one polymeric tubular structure having a stent contacting surface disposed circumferentially about one of said luminal or exterior stent surfaces;
wherein said polymeric tubular structure is made of biological or genetically-engineered spider silk or a derivative thereof.

2. The stent-graft prosthesis of claim 1 further comprising a second polymeric tubular structure having a stent contacting surface disposed circumferentially about the other of said luminal or exterior stent surfaces.

3. The stent-graft prosthesis of claim 2 wherein said second structure is selected from a group of materials consisting of biological or genetically-engineered spider silk and its derivatives.

4. The stent-graft prosthesis of claim 2 wherein said second structure is selected from the group of polymers consisting of polyolefins, polyester, nylon, polyurethane and any combination thereof.

5. The stent-graft device of claim 2 wherein said polymeric tubular structures are sutured to said stent.

6. The stent-graft prosthesis of claim 2 wherein said polymeric tubular structures are secured to one another through said open spaces of said stent.

7. The stent-graft prosthesis of claim 6 wherein said polymeric tubular structures are laminated together through said open spaces of said stent.

8. The stent-graft prosthesis of claim 6 wherein said polymeric tubular structures are adheringly secured through said open spaces of said stent.

9. The stent-graft prosthesis of claim 6 wherein said polymeric tubular structures are sutured to one another through said open spaces of said stent.

10. A method of manufacturing an improved stent-graft composite endovascular prosthesis for preventing endothelial inflammatory responses, comprising the steps of:
providing an elongate radially adjustable stent having a substantially tubular configuration defining a central open passage therethrough, said stent having proximal and distal extremities and opposed interior and exterior stent surfaces wherein said stent includes plural open spaces extending between said opposed luminal and exterior surfaces so as to permit said radial adjustability;
placing a polymeric tubular structure circumferentially about at least one of said luminal and exterior stent surfaces so as to contact a stent surface thereadjacent; and
securing said tubular structure to said stent,
wherein said polymeric structure is made of biological or genetically-engineered spider silk.

11. The method of claim 10 wherein a second polymeric tubular structure having a stent contacting surface is disposed circumferentially about the other of said luminal or exterior stent surfaces not covered by said polymeric tubular structure.

12. The method of claim 11 wherein said second structure is selected from a group of materials consisting of biological or genetically-engineered spider silks and derivatives thereof.

13. The method of claim 11 wherein said second structure is selected from the group of polymers consisting of polyolefins, polyester, nylon, polyurethane and any combination thereof.

14. The method of claim 11 wherein said polymeric tubular structures are sutured to said stent.

15. The method of claim 11 wherein said polymeric tubular structures are secured to one another through said open spaces of said stent.

16. The method of claim 15 wherein said polymeric tubular structures are laminated together through said open spaces of said stent.

17. The method of claim 15 wherein said polymeric tubular structures are adheringly secured through said open spaces of said stent.

18. The method of claim 15 wherein said polymeric tubular structures are sutured to one another through said open spaces of said stent.

19. An implantable tubular prosthesis that minimizes tissue inflammatory responses, comprising:
an expandable tubular structure comprising biological or genetically-engineered spider silk or a derivative thereof, said tubular structure including a tissue contacting outer surface circumferentially defined therearound and an inner blood contacting surface concentric thereto.

20. The implantable tubular prosthesis of claim 19 wherein said prosthesis includes a second polymeric tubular structure disposed circumferentially about either of said tissue contacting outer surface and or inner blood contacting surface.

21. The implantable tubular prosthesis of claim 20 wherein said polymeric tubular structure is formed from a seamless sheet having opposed longitudinal edges and wherein said edges are joined to form said tubular structure.

22. The implantable tubular prosthesis of claim 20 wherein said second structure is selected from a group of materials consisting of biological or genetically-engineered spider silk and its derivatives.

23. The implantable tubular prosthesis of claim 20 wherein said second structure is selected from the group of polymers consisting of polyolefins, polyester, nylon, polyurethane and any combination thereof.

24. The implantable tubular prosthesis of claim 23 wherein said second structure is formed from an extruded tube.

25. The implantable tubular prosthesis of claim 20 wherein said tubular structures are securable to one another.

26. The implantable tubular prosthesis of claim 25 wherein said tubular structures can be laminated together.

27. The implantable tubular prosthesis of claim 25 wherein said tubular structures are adheringly secured to one another.
